(19) United States
(12) Patent Application Publication
(10) Pub. No.: US 2024/0087676 A1
(43) Pub. Date: Mar. 14, 2024

(54) DYNAMIC SAMPLING FOR TUMOR FEATURES AND METABOLITES

(71) Applicant: Protein Fluidics, Inc., Burlingame, CA (US)

(72) Inventors: Evan Francis Cromwell, Redwood City, CA (US); Ekaterina Moroz Nikolov, Snohomish, WA (US); Rashmi Rajendra, Foster City, CA (US); Anthony B Thai, San Bruno, CA (US); Nicholas J Colella, Pleasanton, CA (US)

(21) Appl. No.: 18/464,167

(22) Filed: Sep. 8, 2023

Related U.S. Application Data
(60) Provisional application No. 63/375,178, filed on Sep. 9, 2022.

(51) Int. Cl. G16B 15/30; B01L 3/00

(52) U.S. Cl. CPC G16B 15/30; B01L 3/502753

(57) ABSTRACT

Methods, systems, and computer program products for processing cell samples and training machine learning models are disclosed. Some implementations relate to processing disease cell samples to obtain dynamic response data of the cell samples. Some implementations relate to training machine learning models for mapping therapeutics or stimuli to disease outcomes through dynamic response profiles.

### INCORPORATION BY REFERENCE

An Application Data Sheet is filed concurrently with this specification as part of the present application. Each application that the present application claims benefit of or priority to as identified in the concurrently filed Application Data Sheet is incorporated by reference herein in its entirety and for all purposes.

### BACKGROUND

Diseases are often treated as a static “state function” by clinicians. A starting point is assessed, a therapeutic course is determined, a disease is treated, and an endpoint is measured. If the endpoint is “Positive” then treatment is considered successful. If the endpoint is “Negative” then a different course of treatment can be prescribed. Drug Discovery processes often follow this paradigm. A disease model is selected (e.g., immortalized cells), a matrix of compounds are applied, and an endpoint is measured. These endpoints can be cell viability, biomarker expression, phenotypic changes, target inhibition/promotion, cellular functional changes, etc. This paradigm does not consider dynamic changes to the disease or disease model during treatment such as changes in biomarker expression, metabolism, cell function, resistance to treatment, inflammatory response(s), epigenetic perturbations, etc. Many aspects of disease are dynamic. The dynamics may affect disease outcome in ways that cannot be adequately captured by static measurements. It is desirable to have drug discovery platforms and tools that account for disease dynamics.

### SUMMARY

In one aspect, methods are provided for processing cell samples of a disease model.

In another aspect, methods and systems are provided for training one or more machine learning models.

These and other objects and features of the present disclosure will become more fully apparent from the following description and appended claims, or may be learned by the practice of the disclosure as set forth hereinafter.

### BRIEF DESCRIPTION OF THE DRAWINGS

FIG. 1 illustrates relations among therapy, diseased cells, dynamic transition state of diseased cells, and disease outcome.

FIG. 2A illustrates a computer-implemented disease outcome prediction module that can be used to predict disease outcomes based on candidate therapeutics according to some implementations disclosed herein.

FIG. 2B illustrates a computer-implemented therapeutic prediction module that can be used to predict therapeutics based on desired disease outcomes according to some implementations.

FIG. 3 shows a flowchart of a process for treating cell samples of a disease model and training at least one machine learning model using data obtained from the cell samples.

FIG. 4 illustrates a microfluidic system that may be used in some implementations.

FIG. 5 illustrates an example setup where therapeutic is transferred from a reagent well on the left through a microfluid channel to a sample well at the center.

FIG. 6 shows a flowchart of a process for training machine learning models according to some implementations.

FIG. 7 schematically illustrates an example of training the first forward machine learning model and the second forward machine learning model according to some implementations.

FIG. 8 schematically illustrates an example of using the trained first and second forward machine learning models to predict disease outcomes.

FIG. 9 schematically illustrates an example of training the first backward machine learning model and the second backward machine learning model according to some implementations.

FIGS. 10 and 11 schematically illustrate examples of using the backward machine learning models to predict new therapeutics that are expected to be associated with desired disease outcomes.

FIG. 12 schematically illustrates the mechanism of an artificial neural network (ANN) or simply a neural network according to some implementations.

FIG. 13 illustrates the mechanism of a convolutional neural network (CNN) according to some implementations.

FIG. 14 schematically illustrates the model structure of a generative adversarial network (GAN).

FIG. 15 illustrates an example architecture and some functions of a variational autoencoder that may serve as a feature extractor as described in this section.

FIG. 16 is a block diagram of an example of the computing device or system suitable for use in implementing some embodiments of the present disclosure.

FIG. 17 shows tumoroid formation and flowchip loading.

### DETAILED DESCRIPTION

The disclosed embodiments concern methods, apparatus, and systems for processing cell samples for disease models and training machine learning models.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the Specification as a whole. It is to be understood that this disclosure is not limited to the particular methodology, protocols, and reagents described, as these may vary, depending upon the context they are used by those of skill in the art.

As used herein, the singular terms “a,” “an,” and “the” include the plural reference unless the context clearly indicates otherwise.

### INTRODUCTION

Many aspects of disease are dynamic. Metabolism is a dynamic activity. Long term (days to weeks) changes in metabolism may be studied for response to diet, therapies, hypoxia, etc. Some shorter term (e.g., 1 to 24-hour) responses may also be studied for on-set of metabolic changes. Cancer resistance is a dynamic activity. Acquired resistance to chemotherapy is known and is typically measured over weeks to months. Inflammation response is a dynamic activity. Infected cells will secrete biomolecules (cytokines, chemokines, etc.) that then attract immune cells. Immune cells then interact with those cells to determine a course of action (e.g., recruit other immune cells, kill the cell, let it alone, etc.). Cancer resistance to immuno-therapy is dynamic. Cancer cells will create barriers to immune cell interactions through mimicking healthy cell signaling (PD-1/PD-L1), creating metabolically adverse conditions (Warburg effect), interacting with other cells in the tumor microenvironment to shield cancer cells (cancer associated fibroblasts, tumor associated macrophages, etc.), or other mechanisms. The dynamics may affect disease outcome differently than static responses.

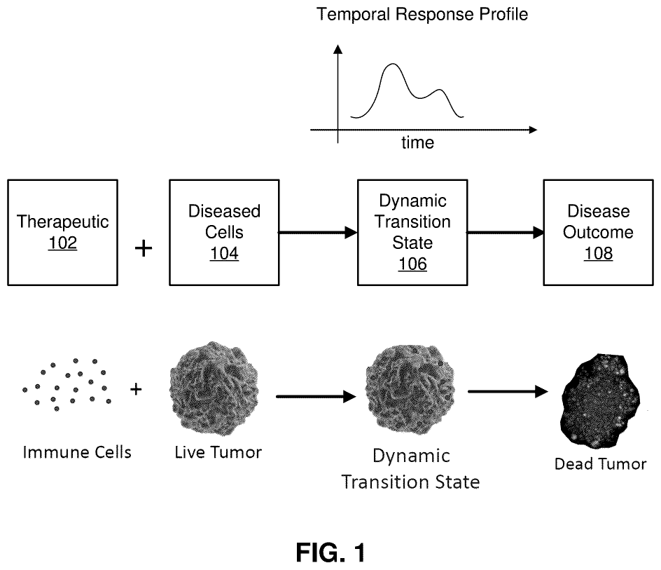

FIG. 1 illustrates relations among therapy, diseased cells, dynamic transition state of diseased cells, and disease outcome. After a therapeutic 102 is applied to diseased cells 104, diseased cells enter a dynamic transition state 106, in which the diseased cells generate one or more temporal response profiles associated with cellular functions and phenotypes. The dynamic transition state of the diseased cells and the temporal response profiles lead to or are associated with the disease outcome 108. At the bottom of the figure, immune cells are illustrated as a form of therapeutic, and tumor cells are illustrated as diseased cells. Tumor dynamic transition state and tumor outcome resulting from treatment are also illustrated. The relations among the therapeutic, the diseased cells, their dynamic transition state, and disease outcome suggest that modeling a disease response to a therapeutic as a static process may miss useful information that affects or is associated with a disease outcome. Instead, modeling disease through the transitional state may provide more accurate prediction of disease outcome.

As an analogy, chemical reactions were viewed as kinetic events going from Starting Material to Output material. They were modeled using Collision Theory as 1st order, 2nd order, etc. based on variations in kinetics based on starting or ending concentrations of the chemical constituents. Disease has been modeled in a similar fashion. In cancer, for example, the starting materials are a tumor and a drug compound. Optimal concentration of the drug compound is determined, and disease is monitored kinetically by output (e.g., dead or live cells). An improved method is to consider that the tumor goes through a Transition State in response to treatment. Dynamic interrogation can elucidate information about this state and lead to improved therapies, such as immuno-therapy in cancer.

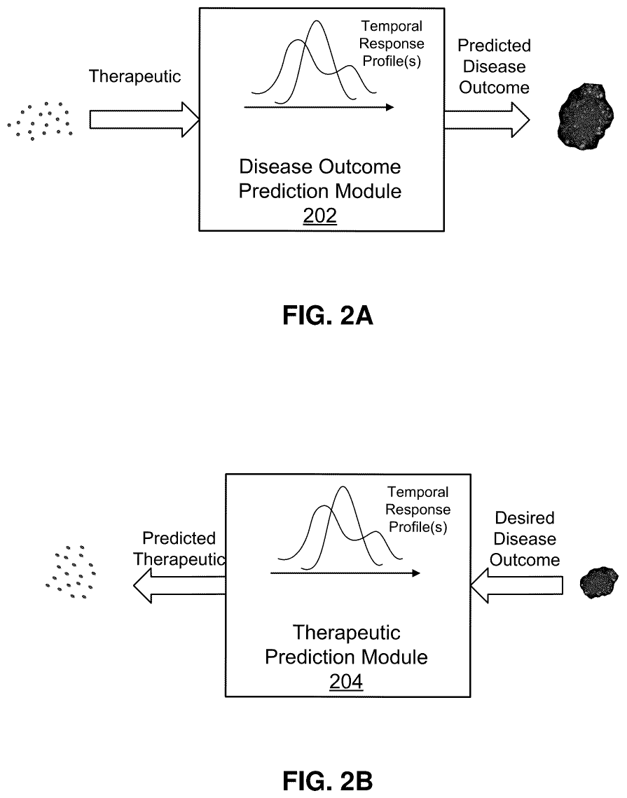

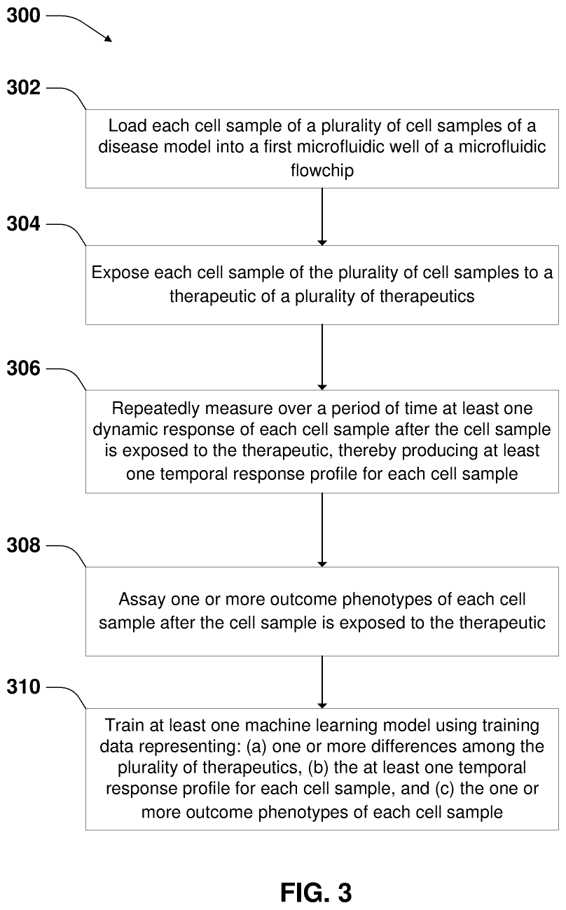

FIG. 2A illustrates a computer-implemented disease outcome prediction module (box 204) that can be used to predict disease outcomes based on candidate therapeutics according to some implementations disclosed herein. FIG. 2B illustrates a computer-implemented therapeutic prediction module (box 204) that can be used to predict therapeutics based on desired disease outcomes according to some implementations. These modules take into consideration the dynamic transition state of diseased cells. In some implementations, they may provide more accurate and valid predictions than conventional methods that do not account for the dynamic responses of cells.

### Methods for Processing Disease Cell Samples and Training Machine Learning Models

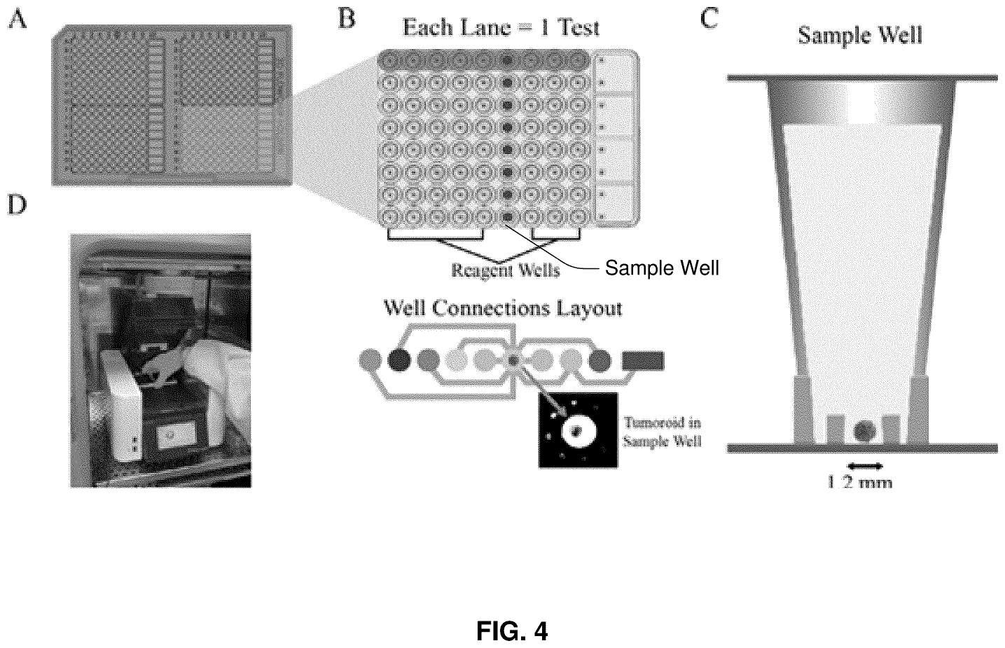

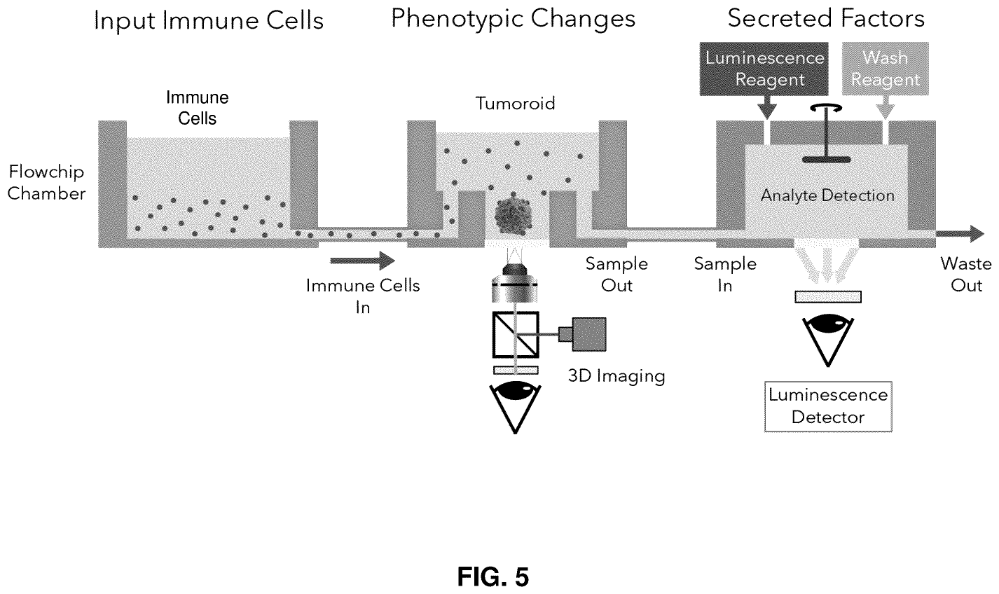

FIG. 3 shows flowchart of a process 300 for treating cell samples of a disease model and training at least one machine learning model using data obtained from the cell samples. Process 300 starts by loading each cell sample of the plurality of cell samples of a disease model into the first microfluidic well of a microfluidic flowchip. See box 302. The microfluidic flowchip includes one or more networks of microfluidic wells connected by microfluidic channels. The microfluidic flowchip is controlled by one or more processors configured to automated fluidic flow in the microfluidic flowchip. In some implementations, loading a cell sample involves coating the cell sample with magnetic nanoparticles and immobilizing the cell sample in the first microfluidic well using a magnet. FIG. 4 illustrates a microfluidic system that may be used in some implementations. Panel A of the figure shows a microfluidic flowchip with four quadrants. Panel B shows an enlarged view of one quadrant of the microfluidic flowchip. In the quadrant, there are eight rows and ten columns of microfluidic wells. Each row is a lane of wells connected by microfluidic channels forming a network. In this example, the sample well is the sixth well from the left. An enlarged view of the sample well is shown in panel C. A cell sample (e.g., a three-dimensional organoid or tumoroid) may be loaded into the sample well by manual or automated methods and then immobilized at the bottom of the sample well. Examples of other microfluidic flowchip and system that may be used are described in U.S. Pat. No. 11,376,589, which is incorporated by reference in its entirety.

In various implementations, the plurality of cell samples includes at least 8 or 32 cell samples. In various implementations, the plurality of cell samples includes 64, 128, 256, 512, or 1024 cell samples. In some implementations, each cell sample includes a three dimensional cell sample. In some implementations, each cell sample includes a tumoroid. In some implementations, the cell sample includes an organoid. In some implementations, the cell sample includes a spheroid, a multicellular spheroid or ellipsoid, or a three-dimensional sample comprising different subtypes.

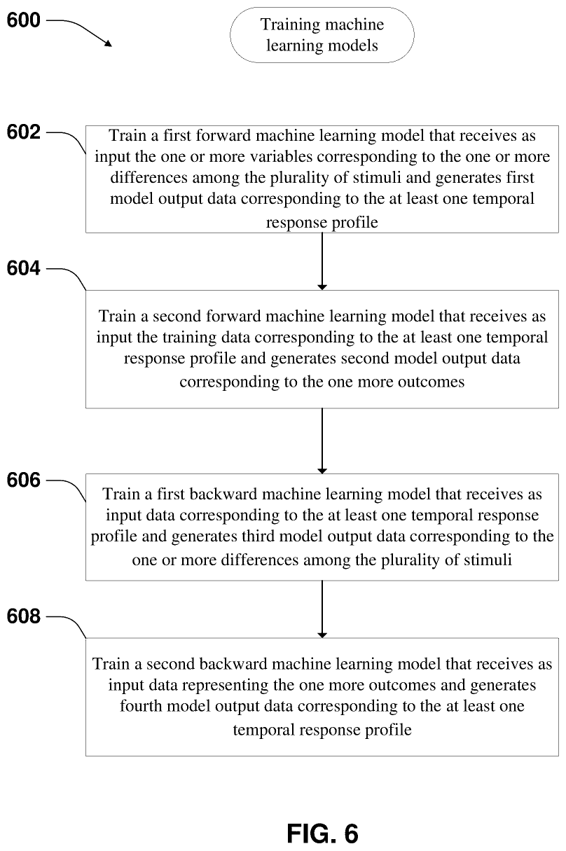

Process 300 further includes exposing each cell sample of the plurality of cell samples to the therapeutic of the plurality of therapeutics. See box 304. Each therapeutic is selected includes one or more compounds, one or more cells, one or more physical conditions, or any combinations thereof. The plurality of therapeutics are different from each other in at least one aspect. In some implementations, each aspect of the at least one aspect is selected from the group consisting of an identity of the therapeutic, the presence of the therapeutic, a structural component of the therapeutic, a functional component of therapeutic, the dosage or concentration of the therapeutic, a time when a therapeutic is applied, and any combinations thereof. In some implementations, exposing each of the plurality of cell samples to the therapeutic of the plurality of therapeutics involves loading each therapeutic into each of second microfluidic wells and transferring each therapeutic from each second microfluidic well to each first microfluidic well. FIG. 5 illustrates an example setup where therapeutic is transferred from a reagent well on the left through a microfluid channel to a sample well at the center. In this example, the therapeutic includes immune cells, and the cell sample includes a tumoroid. After the tumoroid is exposed to the immune cells, supernatants from the sample well in the center is transferred through a microfluidic channel to a microfluidic well on the right, in which analytes are detected and assayed.

Returning to FIG. 3, process 300 further includes repeatedly measuring over a period of time at least one dynamic response of each cell sample after the cell sample is exposed to the therapeutic, thereby producing at least one temporal response profile for each cell sample. See box 306. In some implementations, the at least one dynamic response includes a plurality of dynamic responses and the at least one temporal response profile includes a plurality of temporal response profiles. Each temporal response profile includes a plurality of measurements of the dynamic response obtained at the plurality of points in the period of time. In some implementations, exposing each cell sample of the plurality of cell samples to the therapeutic of the plurality of therapeutics includes loading each therapeutic into each of a second microfluidic wells and transferring each therapeutic from each second microfluidic well to each first microfluidic well. As shown in the example of FIG. 5, supernatants including analytes corresponding to the dynamic response are transferred from the sample well to the reagent well for analysis and detection of analytes associated with the dynamic response. In some implementations, measuring over the period of time at least one dynamic response includes measuring in situ the dynamic response in the first microfluidic well inside the sample well. In some implementations, the plurality of points in the period of time includes at least 4, 10, 20, 30, 40, 50, 60, 70, 80, 90, 100, 200, 500, 1000 points in the period of time. In various implementations, the period of time comprises a period of at least 5, 10, 15, 20, 25, 30, 35, 40, 45, 50 or 55 minutes, 1, 2, 3 4, 5, 6, 7, 8, 9, 12, 15, 18, 21 or 24 hours, or at least 1, 2, 3, 4, 5, 6 or 7 days, or at least 1, 2, 3 or 4 weeks, or at least 1, 2, 3, 4, 5, 6, 7, 8, 9, 10, 11 or 12 months, or at least 1, 2, 3, 4, 5, 6, 7, 8, 9 or 10 years.

Many methods are suitable for dynamically interrogating the dynamic response of the cell samples. Some implementations may use real-time or near real-time sampling of cell supernatants to measure secreted biomolecules. Discrete sampling may improve upon integrated sampling methods as it provides a lower background level and hence higher signal-to-background ratio.

As mentioned above, in some implementations, assays are performed away from the disease model sample and can be done by photonic methods, including luminescence, fluorescence, absorbance, or other methods. Some implementations involves in situ measurement of secreted factors in the vicinity of a disease model sample (e.g., tumoroid, organoid, or cell model). For example, Real-time Glo measures cell secreted molecules indicating cell health. This can be luminescence, fluorescence, absorbance, or other methods.

Some implementations use fluorescence or luminescence probes for biomarkers. These may be engineered to create a signal or reduce/quench a signal in the presence of a biomolecule (e.g., PCR probes). The signal can be optical based, electrical based, or other. A time-dependent change in signal is measured to indicate dynamic changes in biomarker expression.

Some implementations use fluorescence or luminescence molecules indicating cellular function. E.g., calcium sensitive dyes indicating intracellular or extracellular calcium concentrations.

Some implementations use optogenetic probes for cellular function, biomarker expression, secretions. Optogenetic probes can be modulated by presence of light. This can allow timing of interrogation to optimized to a specific time window of dynamic response. One could also use frequency modulation (e.g., lock-in amplifiers) to improve signal-to-noise for low expressions. Some implementations apply transition-state probes that are specific or sensitive to transition state of the cells.

In some implementations the at least one dynamic response in process 300 includes a change in an item selected from a group consisting of: cellular function, biomarker expression, cellular secretion, cellular structure, protein expression, protein cellular localization, protein-protein interaction, cell-cell interaction, cell-extracellular matrix interaction, cell signaling, cell death process, cell viability, and any combinations thereof. In some implementations, the at least one dynamic response includes a change in an item selected from a group consisting of: cytokines, chemokines, growth factors, and any combinations thereof. In some implementations, the at least one dynamic response includes a change in an item selected from a group consisting of: IL-18, IL2, IL4, IL-6, IL8, TGF-b, IL-10, IL-12, IFN-γ, TNF-α, CCR2, CCR5, NF-kB, CXCL2/9/10/11/12, VEGF, and any combinations thereof.

In some implementations, the at least one dynamic response includes a metabolic response. In some implementations, the metabolic response includes a change in an item selected from a group consisting of: lactate, glutamate, glucose, glutamine, lipids, amino acids, bile acids, biogenic amines, carbohydrates, carboxylic acids, fatty acids, hormones, and any combinations thereof.

In some implementations, the at least one dynamic response includes an immune response. In some implementations, the immune response includes a change in an item selected from a group consisting of: cytokines, chemokines, growth factors, Granzyme A and Granzyme B, CTL4, CCR5 perforin, TRAIL, FASL, ROS, NO, and any combinations thereof.

In some implementations, the at least one dynamic response includes a cancer resistance response. In some implementations, cancer resistance response includes a change in an item selected from a group consisting of: cell proliferative index, PD-1 receptor, PD-L1 ligand, other immune checkpoint receptors and ligands, metabolism related pathways, cancer associated fibroblasts, tumor associated macrophages, cell signaling receptors, tumor associated antigens, and any combinations thereof.

In some implementations, the at least one dynamic response comprises a inflammation response.

Process 300 further includes assaying one or more outcome phenotypes of each cell sample after the cell sample is exposed to the therapeutic. See block 308. A phenotype is an observable constitution or appearance of the cell sample. Phenotypes are affected by the genetic composition of cells and environmental impacts including therapeutics. In some implementations, each of the one or more outcome phenotypes is selected from the group consisting of number of cells, number of live cells, number of dead cells, cell proliferative index, apoptosis, integrity of cells, shape of cells, size of cells, size of sample, cell-cell distance, distance between cell types, shape, size, area, volume, perimeter, roundness/circularity of a three-dimensional sample, and any combinations thereof.

Process 300 further includes training at least one machine learning model using training data representing: (a) one or more differences among the plurality of therapeutics, (b) the at least one temporal response profile for each cell sample, and (c) the one or more outcome phenotypes of each cell sample. See block 310. The at least one machine learning model receives as input one or more variables representing the one or more differences in the at least one temporal response profile. The at least one machine learning model provides as output one or more variables representing the one or more outcome phenotypes. In some implementations, the one or more differences among the plurality of therapeutics may be represented as categorical variables representing different categories of therapeutics or components of therapeutics. In some implementations, the differences (e.g., dosage or timing) among the therapeutics may be represented by continuous variables. In various implementations, the differences among the therapeutics are represented by variables that may be adjusted and/or combined in ways that correspond to different therapeutics. In other words, the differences among the therapeutics are parameterized, which provides a therapeutic space in which different therapeutics may be explored systematically.

In some implementations, the at least one machine learning model includes one or more models selected from a group consisting of: a neural network, a convolutional neural network (CNN), an autoencoder, a variational autoencoder (VAE), a regression model, a linear model, a non-linear model, a support vector machine, a decision tree model, a random forest model, an ensemble model, a Bayesian model, a naïve Bayes model, a k-means model, a k-nearest neighbors model, a principal component analysis, a Markov model, and any combinations thereof.

In some implementations, the at least one machine learning model includes a first machine learning model that receives as input one or more variables representing the one or more differences and provides as output at least one variable representing the at least one temporal response profile, and a second machine learning model that receives as input the variable representing the temporal response profile and provides as output the one or more variables representing the one or more outcome phenotypes.

In some implementations, the at least one machine learning model includes a machine learning model that receives as input one or more variables representing the one or more differences and the at least one temporal response profile and provides as output one or more variables representing the one or more outcome phenotypes.

In some implementations, the at least one machine learning model generates one or more intermediate variables from model input and predicts output using the one or more intermediate variables. In some implementations, each of the one or more intermediate variables is selected from a group consisting of: a variable representing the at least one temporal response profile, a T-cell Functional Response Score (TFRS), a tumor response score, a cell type specific response score, a therapeutic treatment response score, a therapeutic sensitivity score, a therapeutic resistance score, a latent variable, a first derivative of the temporal response profile, a second derivative of the temporal response profile, an IC50 value, an EC50 value, a transition state expression transient, and any combinations thereof. In some implementations, a first machine learning model of the at least one machine learning model generates one or more intermediate variables as output, and a second machine learning model of the at least one machine learning model receives as input the one or more intermediate variables and provides as output the one or more variables representing the one or more outcome phenotypes.

In some implementations not shown in FIG. 3, process 300 further includes: providing values of the one or more variables representing the one or more differences for one or more new therapeutics to the at least one trained machine learning model to predict the one or more outcome phenotypes for the one or more new therapeutics, wherein the one or more new therapeutics are different from the plurality of therapeutics. In some implementations not shown in FIG. 3, process 300 further includes exposing a cell sample of the model of the disease to a new therapeutic predicted to result in values of the one or more outcome phenotypes meeting one or more criteria. In some implementations not shown in FIG. 3, the process further includes administering the new therapeutic to a patient having or suspected of having the disease.

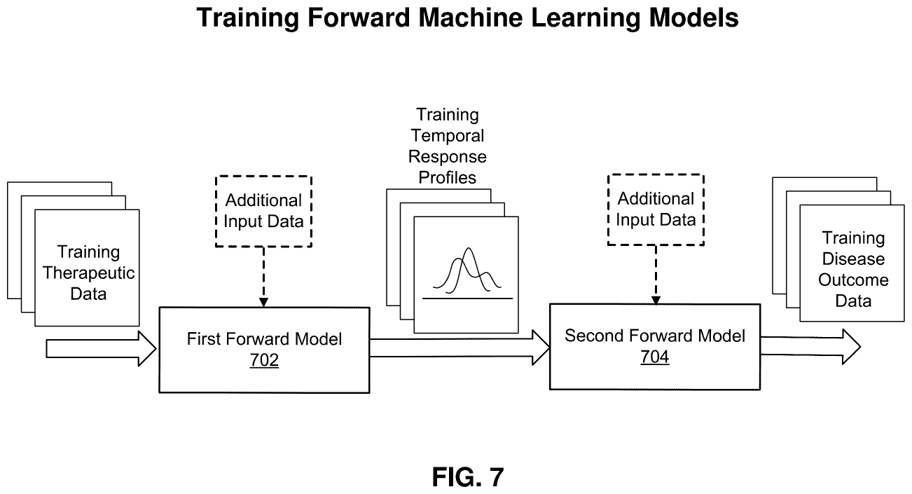

The operation of block 310 in FIG. 3 may be performed according to a process 600 in FIG. 6 in some implementations. FIG. 6 shows a flowchart of process 600 for training machine learning models according to some implementations. Process 600 involves training a first forward machine learning model that receives as input the one or more variables corresponding to the one or more differences and generates first output data corresponding to the at least one temporal response profile. See block 602. The terms “forward” and “backward” are used to indicate the information flow direction of the machine learning models. In a forward model, information flows in a direction from therapeutics to disease outcome via various cellular responses. Conversely, a backward machine learning model has an information flow in the reverse direction going from disease outcome to therapeutics. In various implementations, each of the machine learning models of process 600 is selected from: a neural network, a convolutional neural network (CNN), an autoencoder, a variational autoencoder (VAE), a regression model, a linear model, a non-linear model, a support vector machine, a decision tree model, a random forest model, an ensemble model, a Bayesian model, a naïve Bayes model, a k-means model, a k-nearest neighbors model, a principal component analysis, a Markov model, and any combinations thereof.

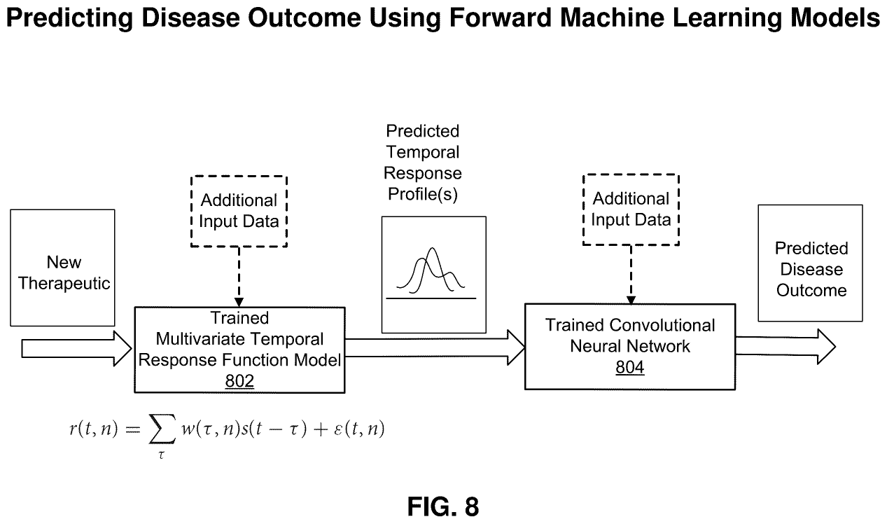

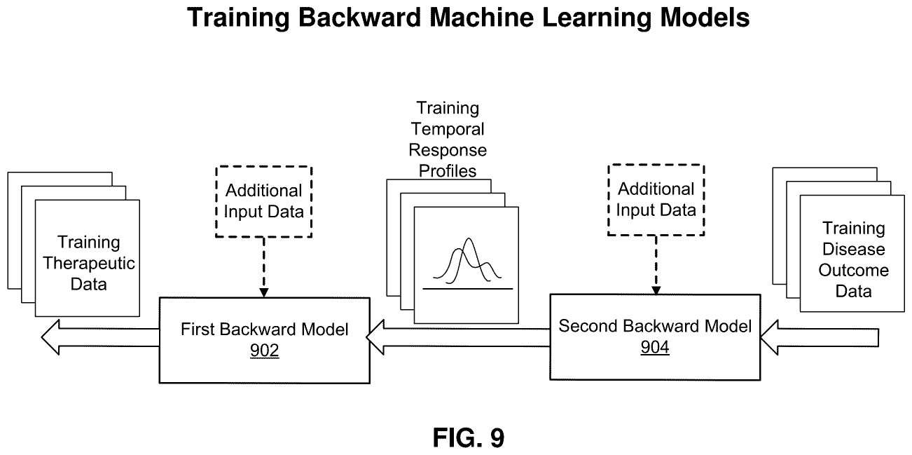

Process 600 further includes training a second forward machine learning model that receives as input the training data corresponding to the at least one temporal response profiles and generates second model output data corresponding to the one or more disease outcomes. See block 604. FIG. 7 schematically illustrates an example of training the first forward machine learning model and the second forward machine learning model according to some implementations. FIG. 8 schematically illustrates an example of using the trained first and second forward machine learning models to predict disease outcomes.

Process 600 further includes training a first backward machine learning model that receives as input data corresponding to the at least one temporal response profile and generates third model output data corresponding to the one or more differences among the plurality of stimuli. See block 606.

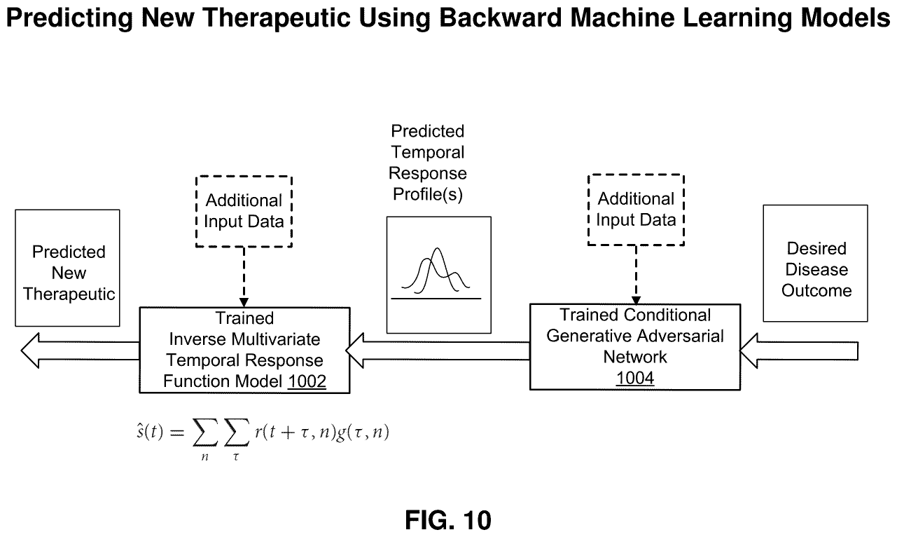

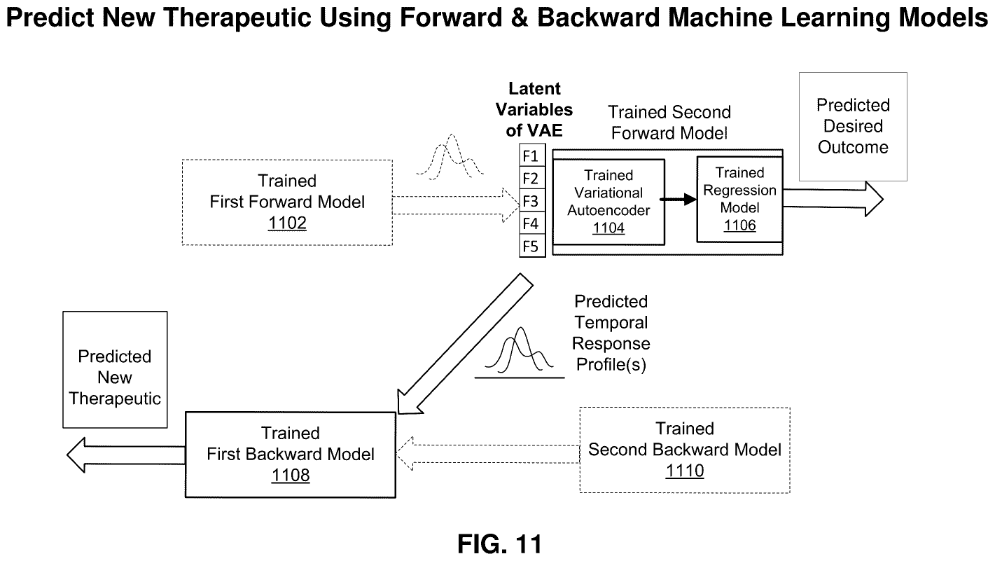

Process 600 further includes training the second backward machine learning model that receives as input data representing the one or more outcomes and generates fourth model output data corresponding to the at least one temporal response profile. See block 608. FIG. 9 schematically illustrates an example of training the first backward machine learning model and the second backward machine learning model according to some implementations. FIGS. 10 and 11 schematically illustrate examples of using the backward machine learning models to predict new therapeutics that are expected to be associated with desired disease outcomes.

In some implementations, process 600 may be used to train machine learning models that relate to therapeutics and disease outcome. In some implementations, the plurality of stimuli of process 600 corresponds to a plurality of different therapeutics. In some implementations, the outcome of process 600 relates to disease outcomes.

FIG. 7 schematically illustrates a process for training a first forward machine learning model 702 and a second forward machine learning model 704. The first forward machine learning model 702 receives input data corresponding to different therapeutics and outputs temporal response profiles associated with the therapeutics. The second forward machine learning model 704 receives as input temporal response profiles generated by cell samples in response to therapeutics. The second forward machine learning model 704 provides as output disease outcomes. In the training process, training samples preferably from different parts of the sample data space are used to train the models to establish mapping between model input and model output. In some implementations, the first forward machine learning model includes a regression model or a neural network model. In some implementations, the regression model includes a multivariate temporal response function model (mTRF model). The mTRF model includes independent variables corresponding to the one or more differences among the plurality of therapeutics. In some implementations, the neural network model includes a conditional generative adversarial network (cGAN). The cGAN includes one or more conditions corresponding to the one or more differences among the plurality of therapeutics. In some implementations, the first or second forward machine learning models may optionally receive as further input one or more static response variables measured at one point of time after the cell sample is exposed to the therapeutic. The additional input data are illustrated in dash lines, indicating that they are optional. In some implementations, the first or second forward machine learning model may receive as further input one or more pre-therapeutic variables corresponding to measurements of the cell sample taken before the cell sample is exposed to the therapeutic. In other implementations, the pre-therapeutic variables may be used to standardize other variables or provide baselines for other variables. In some implementations, the pre-therapeutic variables may correspond to various phenotypes of the cell samples before treatment.

In some implementations, the first forward machine learning model 702 generates one or more intermediate variables (not shown in FIG. 7), and the second forward machine learning model uses the intermediate variables to predict disease outcomes. In some implementations, each of the intermediate variables is selected from a group consisting of: a variable representing the at least one temporal response profile, a T-cell Functional Response Score (TFRS), a tumor response score, a cell type specific response score, a therapeutic treatment response score, a therapeutic sensitivity score, a therapeutic resistance score, a latent variable, a first derivative of the temporal response profile, a second derivative of the temporal response profile, an IC50 value, an EC50 value, a transition state expression transient, and any combinations thereof. These intermediate variables may be measured at different points of time. In various implementations, they are measured before the outcome of the disease occurs or is measured. For example, tumor resistance may be measured in a first time period, which may then be used to predict tumor outcome at a later time using the second forward machine learning model 704. The intermediate variables may be used to train both the first forward machine learning model and the second forward machine learning model. In various implementations, these variables and scores may be derived from other variables or measurements.

FIG. 8 schematically illustrates a process for predicting disease outcomes using the trained forward machine learning models. In this example, the first forward machine learning model is a multivariate temporal response function (mTRF) model 802 as further explained below. The second forward machine learning model is a convolutional network 804 as further explained below.

The mTRF model 802, receives as input a new therapeutic that has not been assayed and/or is different from the training data. The model predicts one or more temporal response profiles for the new therapeutic.

The TRF is a univariate regression model, in which n temporal response functions are recorded by N channels. It assumes that an instantaneous response r(t, n), sampled at times t=1 . . . T and at channel n, is provided by a convolution of the stimulus property, s(t), with an unknown channel-specific TRF, w(τ, n). The response model can be represented in discrete time according to equation (1):

$$r ( t , n ) = {\sum }_{τ} w ( τ , n ) s ( t - τ ) + ε ( t , n )$$

where ε(t, n) is the residual response at each channel not explained by the model. A TRF can be thought of as a filter that describes the linear transformation of the ongoing therapeutic stimulus to the ongoing cellular response. The TRF, w(τ, n), describes this transformation from stimulus s to response r for a specified range of time lags, τ, relative to the instantaneous occurrence of the stimulus feature, s(t). The range of time lags over which to calculate w(τ, n) might be that typically used to capture a cellular response to a therapeutic, such as a range determined by empirical pharmacokinetics data. The TRF, w(τ, n), is estimated by minimizing the mean-squared error (MSE) between the actual temporal response profile, r(t, n), and that predicted by the convolution, \hat{r}(t, n), according to equation (2):

$$min ε ( t , n ) = {\sum }_{τ} {[ r ( t , n ) - \hat{r} ( t , n ) ]}^{2}$$

The univariate TRF model receives a single stimulus variable and provides a temporal response profile in each channel of N channels. If multiple stimuli are encoded as multiple independent variables, a multivariate TRF (mTRF) model can be applied.

The trained convolutional neural network 804 of FIG. 8 takes as input the temporal response profiles predicted by the mTRF model 802. The train convolutional neural network model then predicts a disease outcome based on the temporal response profiles. In some implementations, a very large number of new therapeutics may be applied to the trained models to obtain predicted disease outcomes. Therefore, new therapeutics that have desired disease outcomes may be identified among the large number of new therapeutics. In some implementations, 10, 100, 1,000, 10,000, 100,000, or 1 million new therapeutics may be virtually screened in silico using this method.

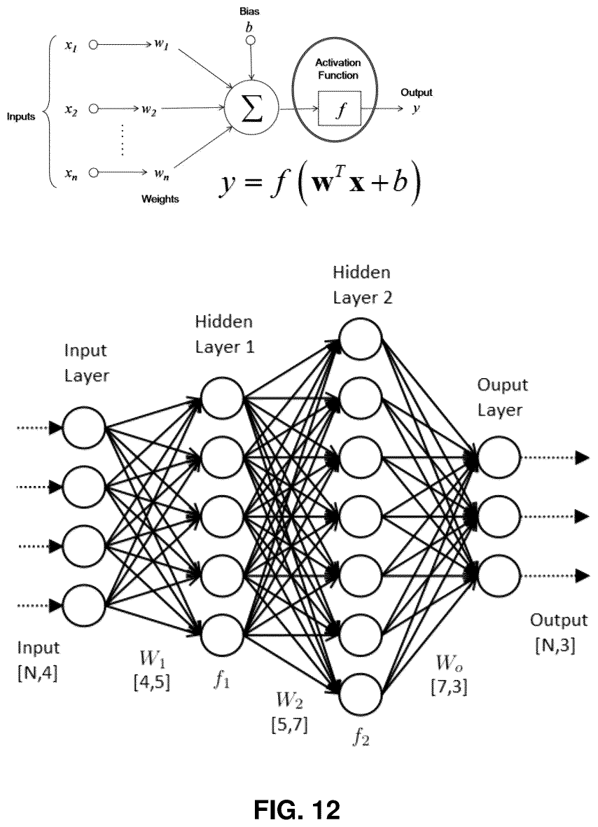

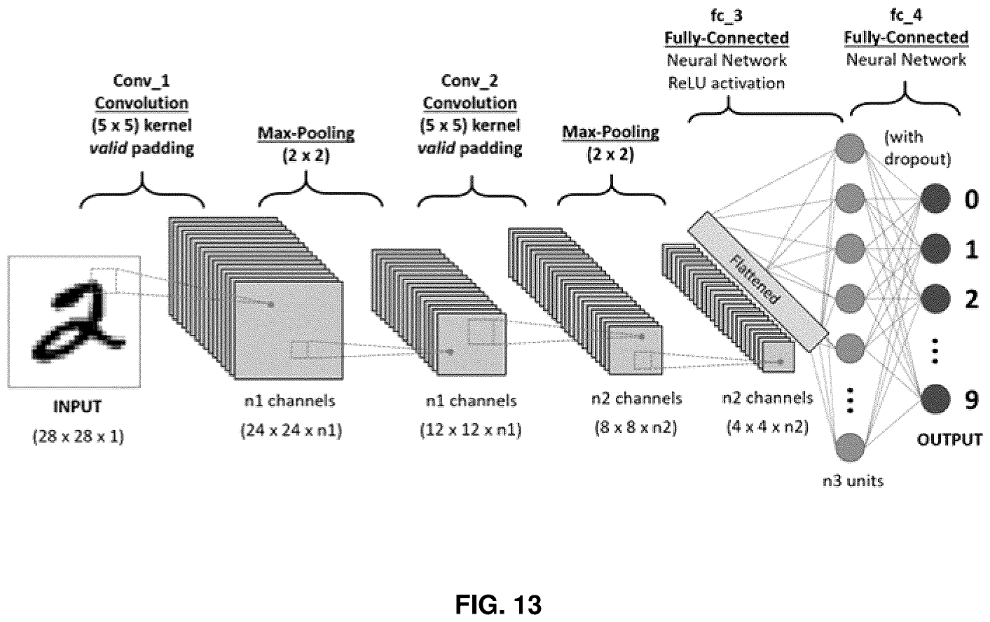

FIG. 12 schematically illustrates the mechanism of an artificial neural network (ANN) or simply a neural network according to some implementations. FIG. 13 illustrates the mechanism of a convolutional neural network (CNN) according to some implementations. The neural network of FIG. 12 corresponds to the fully connected neural network at the right hand side of FIG. 13. The neural network in FIG. 12 is fully connected, such that each neuron or node in one layer of the network connects has a feed forward connection with every neuron in the next layer of the neural network. The connections in the network are unidirectional going in the direction from the input layer to the output layer. The neural network has one input layer and one output layer, with two hidden layers between the input layer and the output layer. Neural networks having more than one hidden layer are also called deep neural network.

Each neuron in the input layer receives an input signal corresponding to an element in an input vector. In classifier neural networks, each neuron in the output layer may represent a class. In the generative neural network, each neuron in the output layer may represent a value in the output data space (e.g., a pixel intensity in an image). For example, if a neural network is configured to distinguish two disease outcome classes, e.g., disease vs. no disease, two output neurons may be used to represent the two disease outcomes. For another example, a neural network may be configured to output, e.g., 10 levels of severity of disease outcome. Here 10 output neurons may be used to represent the 10 levels of disease severity. In these implementations, the final outputs of the neural networks are categorical. The activation functions for the output neurons of these categorial networks may be SoftMax functions in some implementations, resulting in a probability for each neuron, where all probabilities of the output neurons sum up to 1. Their loss functions may cross entropy functions in some implementations. In other implementations, a neural network may be designed to output a continuous variable. For example, it may be desirable to model number of cancer cells killed by a therapeutic. In such implementations, the activation function of an output neuron may be a linear function, a sigmoid function, or another nonlinear function. The loss function may be root square error in some implementations.

The illustration in the top half of FIG. 12 shows the input and output of a neuron of the neural network. The output y of a neuron is determined by the input of its upstream neurons weighted by the strength of the connections w between neurons. The weighted sum of the input from the upstream neurons is combined with a bias term b that ensures a neuron is activated to some degree. Then the combined weighted sum and bias go through an activation function ƒ to provide the output of the neuron y.

Conventional artificial neural network as illustrated in FIG. 12 are not optimal for detecting spatial relation that are invariant across size, location, or orientation. Convolutional neural networks are better able capture such spatial relation and achieve size, location, and orientation invariance, convolutional neural networks are better able to capture such spatial relations. FIG. 13 shows an architecture of convolutional neural network (CNN) according to some implementations. It includes as the input layer a first convolutional layer that uses a 5×5 kernel with a stride size of 1 and n1 channels (for n1 feature maps). The convolutional layer extracts spatial feature in each 5×5 kernel and generates a feature map for each channel. The CNN includes a first max-pooling layer that uses a 2×2 kernel to downscale the future maps generated by the first convolution of layer. The CNN also includes a second convolutional layer that uses a 5×5 kernel with a stride size of n1 and n2 channels. The CNN also includes a second max-pooling layer using a 2×2 kernel to further downscale the future maps generated by the second convolutional later. The further downscaled future maps are then flattened to provide a vector, which becomes the input to the first fully connected layer that is analogous to the ANN of FIG. 12. The CNN may be more effective than a conventional ANN in extracting features from temporal response profiles that have complex patterns and inter-profile relations in some implementations.

FIG. 9 schematically illustrates an example of training the first and second backward machine learning models according to some implementations. The first backward machine learning model 902 receives temporal response profiles as input and provides data corresponding to therapeutics as output. The second backward machine learning model 904 receives as input disease outcome data and provide as output temporal response profiles associated with the disease outcome data. In some implementations, the first backward machine learning model includes a regression model or the neural network model. In some implementations, the regression model includes an inverse multivariate temporal response function (inverse mTRF) model. In some implementations, the neural network model includes a convolutional neural network. In some implementations, the second backward machine learning model includes a regression model. In some implementations, the second backward machine learning model includes a conditional generative adversarial network (cGAN).

In some implementations, the first backward machine learning model 902 receives as further input one or more static response variables each corresponding to a static response of a cell sample measured at one point of time after the cell sample is exposed to the therapeutic. This further input is illustrated as additional input data with dash lines, indicating the input is optional. In some implementations, the first backward machine learning model receives as further input one or more pre-therapeutic variables each corresponding to a measurement of a cell sample taken before the cell sample is exposed to the therapeutic. In some implementations, the first backward machine learning model generates as further output one or more static response variables each corresponding to a static response of a cell sample measured at one point of time after the cell sample is exposed to the therapeutic (not illustrated in FIG. 9). In some implementations, the first backward machine learning model 902 generates as further output one or more pre-therapeutic variables each corresponding to a measurement of cell sample taken before the cell sample is exposed to the therapeutic (not illustrated in FIG. 9).

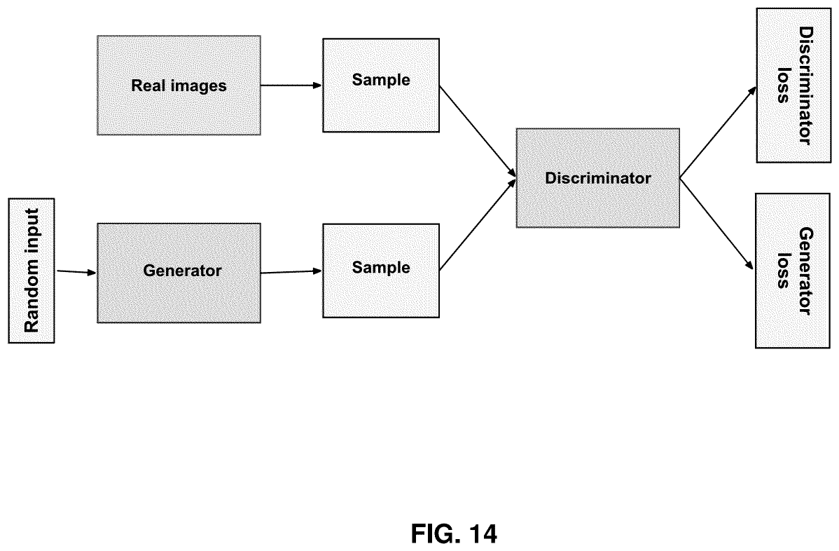

FIG. 10 schematically illustrates a process for predicting new therapeutics using the first and second backward machine learning models (trained) according to some implementations. The first backward machine learning model in the figure includes a trained inverse multivariate temporal response function (inverse mTRF) model 1002 as further explained below. The second backward machine learning model includes a trained conditional generative adversarial network (cGAN) 1004. In some implementations, the cGAN has a model structure as illustrated in FIG. 14 below. The cGAN receives as input a desired disease outcome and predicts one or more temporal response profiles associated with the desired disease outcome. The trained inverse mTRF model 1002 receives as input the one or more temporal response profiles predicted by the trained cGAN 1004. Based on the input, the trained inverse mTRF model 1002 predicts a new therapeutic that is associated with the predict temporal response profiles and the desired disease outcome. In some implementations, this process may be repeated a large number of times to predict new therapeutics associated with desired disease outcomes.

The inverse mTRF is an inverse function of the mTRF, also referred to as a decoder. Its univariate function may be used to predict a therapeutic or stimulus based on a single temporal response profile. Decoders can be modeled in much the same way as TRFs. Suppose the decoder, g(τ, n), represents the linear mapping from the temporal response, r(t, n), back to the therapeutic or stimulus, s(t). This could be expressed in discrete time according to equation (3):

$$\hat{s} ( t ) = {\sum }_{n} {\sum }_{τ} r ( t + τ , n ) g ( τ , n )$$

where ŝ(t) is the reconstructed stimulus property. Here, the decoder integrates the cellular temporal response over a specified range of time lags τ. Ideally, these lags will capture the window of cellular temporal response data that optimizes reconstruction of the stimulus property. Typically, the most informative lags for reconstruction are commensurate with those used to capture the major components of a forward TRF, except in the reverse direction as the decoder effectively maps backwards in time.

The decoder, g(τ, n), is estimated by minimizing the mean squared error (MSE) between s(t) and ŝ(t) according to equation (4):

$$min ε ( t ) = {\sum }_{τ} {[ s ( t ) - \hat{s} ( t ) ]}^{2}$$

FIG. 14 schematically illustrates the model structure of a generative adversarial network (GAN). The GAN includes a generator neural network that generates data mimicking real data such as images. The generator uses random noise as input, which provides a stochastic mechanism to the generated data so that they are analogous to but not identical to real data. The data generated by the generator (e.g., generated images) and real data (e.g., real images) are used as labeled data to train a discriminator neural network. The discriminator improves its ability to discriminate the real images and the generated images through training and its loss function is configured to increase the discriminator's ability to discriminate between the real images and the generated images. A complementary loss function is used to train the generator. As a result, as the generator is initially poor at generating images similar to real images, the discriminator is penalized less and the generator is penalized more. As the generator gets better, the discriminator is penalized more and the generator less. The model stabilizes when the discriminator is able to discriminate about 50% of the time.

A conditional GAN allows the generator to generate images that meets a condition, e.g., the gender of a person of an image. In some implementations, such conditioning may be achieved by labeling the training samples according to the conditions and encoding the conditions as one or more variables in the inputs to the generator. For example, disease outcomes may be divided into different conditions (good or poor) and the training data may be also divided according to the different conditions (good or poor). The conditional GAN's input to the generator may include one or more features encoding the conditions of the training data. During data generation, by specifying input feature corresponding to a condition (good outcome or poor outcome), the generator generates temporal response profiles meeting the condition (good or poor outcome).

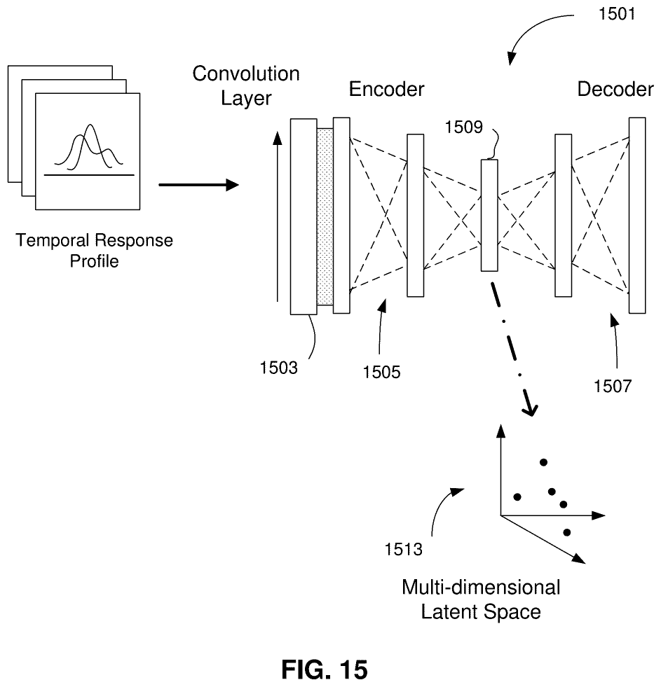

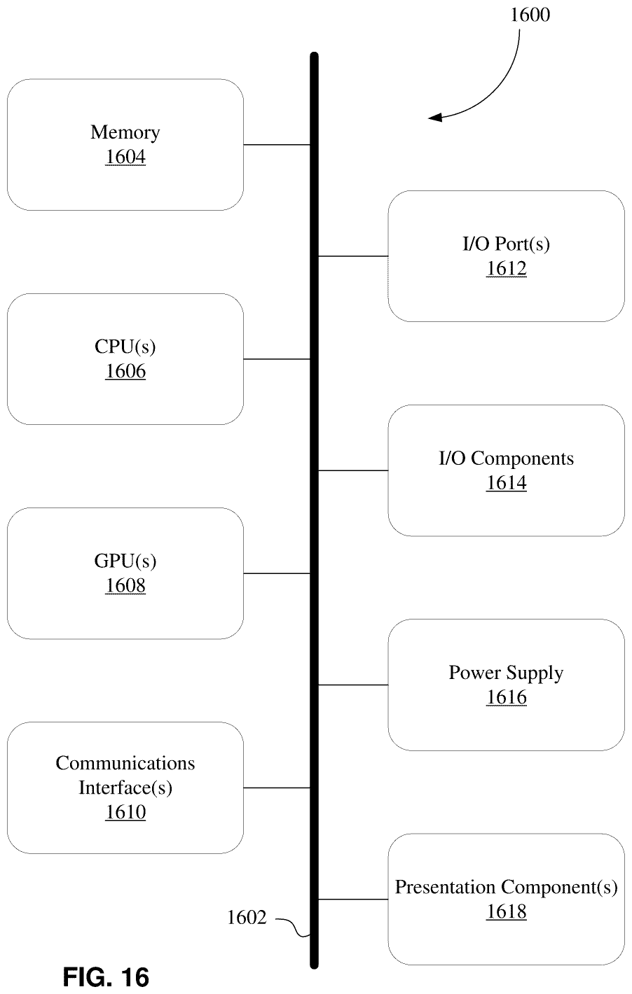

FIG. 11 schematically illustrates a process for predicting therapeutic using both forward and backward machine learning models according to some implementations. In this example, the process uses a trained second forward model (1104 and 1106) and a trained first backward model 1108. The trained second forward model includes a trained variational auto encoder (VAE) 1104 and a trained regression model 1106. The variational auto encoder 1104 is a neural network model that includes an encoder network for encoding input into latent variables in a latent space and a decoder network for decoding and reconstructing data from latent variables as further explained hereinafter with reference to FIGS. 15 and 16. The variational autoencoder is an unsupervised learning model that can extract features, reduced dimensions. It may also be used to generate data that is analogous to but different from its training data. The difference is derived from the stochastic sampling of the latent variables. The process of FIG. 11 does not need to apply the trained first forward model 1102 or the train second backward model 1110, which are shown in dahs lines. The process uses latent variables of the trained VAE 1104 as input to the trained regression model 1106. The trained regression model 1106 predicts the outcome from the latent variables. By examining a number of sets of latent variables, one can identify a set of latent variables predicted to have desired outcomes. After the set of latent variables associated with desired outcome is identified, the process then uses the decoder of the VAE 1104 to generate temporal response profiles from the latent variables. And then the generated temporal response profiles become the input to the trained first backward model 1108. The trained first backward model 1108 then predicts a new therapeutic that is associated with the generated temporal response profiles and desired disease outcome.

FIG. 15 illustrates an example architecture and some functions of a variational autoencoder that may serve as a feature extractor as described in this section. As illustrated, a variational autoencoder 1501 optionally includes a convolution layer 1503 at an input side, a multilayer encoder portion 1505, a multilayer decoder portion 1507, and a hidden or latent space portion 1509. Variational autoencoder 1501 is configured to receive input data such as temporal response profiles 1511 acquired from a test sample. Optionally, the input data is organized and provided to the convolution layer 1503 that is configured to extract potentially relevant features from the spectra. Variational autoencoder 1501 is configured such that the input data filtered by convolution layer 1503 is processed by the encoder layers 1505 and decoded by the decoder layers 1507. Between the encoder and decoder portions is a hidden layer 1509 configured to hold the fully encoded data in a latent space. Variational autoencoder 1501 is an unsupervised learning model, as it can train the model using unlabeled data by comparing the unencoded input and the decoded output to provide a loss function.

In the depicted embodiment, the hidden or latent space portion 1509 holds a multi-dimensional latent space representation 1513 of the fully encoded data. The latent space representation 1513 comprises multiple data points, each associated with a particular sample or a particular reading taken from a sample. In a conventional autoencoder, data in the hidden layer are static. In a variational autoencoder, data in the hidden layer are associated with a random noise sampled from a distribution such as a Gaussian distribution, providing stochastic variation to the system. As such, encoded and decoded data may be different, allowing generation of new data that is analogous but different from training data. Therefore, variational autoencoders may be used as generative models in some implementations.

Each data point in the latent space comprises a feature vector, which has fewer dimensions than input and output data of the autoencoder. Therefore, autoencoders may also be used to extract features, reduce dimensions, and/or reduce noise. The extracted features with lower dimensions may be used as input to other machine learning models such as ANN or regression models.

Training of a VAE may employ loss functions and/or other techniques that project input data to a latent space in a probabilistic manner. In some implementations, the loss functions may employ a regularization term utilizing Kullback-Leibler divergence. The feature extractor projects data not as discrete values but as distributions of values on axes in latent space. The distributions may be characterized by, e.g., their central tendencies (means, medians, etc.) and/or their variances in the latent space. The training may encourage the learned distribution (in latent space) to be similar to the true prior distribution (the input data).

In various implementations, different methods for training machine learning models may be applied, including unsupervised self-supervised, semi-supervised, and supervised learning methods. In certain embodiments, a model such as a VAE is trained in a semi-supervised fashion that employs both labeled and unlabeled training data. Examples of semi-supervised training techniques are described in Yang, X., Song, Z., King, I., & Xu, Z. (2021). A Survey on Deep Semi-supervised Learning. http://arxiv.org/abs/2103.00550, which is incorporated herein by reference in its entirety. In some embodiments, a model is trained in one or more iterations, and in fact may employ multiple separate machine learning models, some serving as a basis for transfer learning of later developed refinements or versions of the model. In some embodiments, a feature extractor is partially trained using supervised learning and partially trained using unsupervised learning.

In some embodiments, learning is conducted in multiple stages using multiple training data sources via a mechanism such as transfer learning. Transfer learning is a training process that starts with a previously trained model and adopts that model's architecture and current parameter values (e.g., previously trained weights and biases) but then changes the model's parameter values to reflect a new or different training data. In various embodiments, the original model's architecture, including convolutional windows, if any, and optionally its hyperparameters, remain fixed through the process of further training such as via transfer learning.

In certain embodiments, one or more training routines produce a first trained preliminary machine learning model. Once fully trained with training data, the preliminary model may be used as a starting point for, e.g., training a second machine learning model. The training of the second model starts by using a model having the architecture and parameter settings of the first trained model but refines the parameter settings by incorporating information from additional training data.

### Computer Systems

FIG. 16 is a block diagram of an example of the computing device or system 1600 suitable for use in implementing some embodiments of the present disclosure. For example, device 1600 may be suitable for implementing some or all operations of training and using machine learning models as disclosed herein.

Computing device 1600 may include a bus 1602 that directly or indirectly couples the following devices: memory 1604, one or more central processing units (CPUs) 1606, one or more graphics processing units (GPUs) 1608, a communication interface 1610, input/output (I/O) ports 1612, input/output components 1614, a power supply 1616, and one or more presentation components 1618 (e.g., display(s)). In addition to CPU 1606 and GPU 1608, computing device 1600 may include additional logic devices that are not shown in FIG. 16, such as but not limited to an image signal processor (ISP), a digital signal processor (DSP), a deep learning processor (DLP), an ASIC, an FPGA, or the like.

Although the various blocks of FIG. 16 are shown as connected via the bus 1602 with lines, this is not intended to be limiting and is for clarity only. For example, in some embodiments, a presentation component 1618, such as a display device, may be considered an I/O component 1614 (e.g., if the display is a touch screen). As another example, CPUs 1606 and/or GPUs 1608 may include memory (e.g., the memory 1604 may be representative of a storage device in addition to the memory of the GPUs 1608, the CPUs 1606, and/or other components). In other words, the computing device of FIG. 16 is merely illustrative. Distinction is not made between such categories as “workstation,” “server,” “laptop,” “desktop,” “tablet,” “client device,” “mobile device,” “hand-held device,” “electronic control unit (ECU),” “virtual reality system,” and/or other device or system types, as all are contemplated within the scope of the computing device of FIG. 16.

Bus 1602 may represent one or more busses, such as an address bus, a data bus, a control bus, or a combination thereof. The bus 1602 may include one or more bus types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus.

Memory 1604 may include any of a variety of computer-readable media. The computer-readable media may be any available media that can be accessed by the computing device 1600. The computer-readable media may include both volatile and nonvolatile media, and removable and non-removable media. By way of example, and not limitation, the computer-readable media may comprise computer-storage media and/or communication media.

The computer-storage media may include both volatile and nonvolatile media and/or removable and non-removable media implemented in any method or technology for storage of information such as computer-readable instructions, data structures, program modules, and/or other data types. For example, memory 1604 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 1600. As used herein, computer storage media does not comprise signals per se.

The communication media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the communication media may include wired media such as a wired network or direct-wired connection, and wireless media such as acoustic, RF, infrared and other wireless media. Combinations of any of the above should also be included within the scope of computer-readable media.

CPU(s) 1606 may be configured to execute the computer-readable instructions to control one or more components of the computing device 1600 to perform one or more of the methods and/or processes described herein. CPU(s) 1606 may each include one or more cores (e.g., one, two, four, eight, twenty-eight, seventy-two, etc.) that are capable of handling a multitude of software threads simultaneously. CPU(s) 1606 may include any type of processor and may include different types of processors depending on the type of computing device 1600 implemented (e.g., processors with fewer cores for mobile devices and processors with more cores for servers). For example, depending on the type of computing device 1600, the processor may be an ARM processor implemented using Reduced Instruction Set Computing (RISC) or an x86 processor implemented using Complex Instruction Set Computing (CISC). Computing device 1600 may include one or more CPUs 1606 in addition to one or more microprocessors or supplementary co-processors, such as math co-processors.

GPU(s) 1608 may be used by computing device 1600 to render graphics (e.g., 3D graphics). GPU(s) 1608 may include many (e.g., tens, hundreds, or thousands) of cores that are capable of handling many software threads simultaneously. GPU(s) 1608 may generate pixel data for output images in response to rendering commands (e.g., rendering commands from CPU(s) 1606 received via a host interface). GPU(s) 1608 may include graphics memory, such as display memory, for storing pixel data. The display memory may be included as part of memory 1604. GPU(s) 1608 may include two or more GPUs operating in parallel (e.g., via a link). When combined, each GPU 1608 can generate pixel data for different portions of an output image or for different output images (e.g., a first GPU for a first image and a second GPU for a second image). Each GPU can include its own memory or can share memory with other GPUs.

In examples where the computing device 1600 does not include the GPU(s) 1608, the CPU(s) 1606 may be used to render graphics.

Communication interface 1610 may include one or more receivers, transmitters, and/or transceivers that enable computing device 1600 to communicate with other computing devices via an electronic communication network, included wired and/or wireless communications. Communication interface 1610 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet), low-power wide-area networks (e.g., LoRaWAN, SigFox, etc.), and/or the internet.

I/O ports 1612 may enable the computing device 1600 to be logically coupled to other devices including I/O components 1614, presentation component(s) 1618, and/or other components, some of which may be built in to (e.g., integrated in) computing device 1600. Illustrative I/O components 1614 include a microphone, mouse, keyboard, joystick, track pad, satellite dish, scanner, printer, wireless device, etc. I/O components 1614 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of computing device 1600. Computing device 1600 may be include depth cameras, such as stereoscopic camera systems, infrared camera systems, RGB camera systems, touchscreen technology, and combinations of these, for gesture detection and recognition. Additionally, computing device 1600 may include accelerometers or gyroscopes (e.g., as part of an inertia measurement unit (IMU)) that enable detection of motion. In some examples, the output of the accelerometers or gyroscopes may be used by computing device 1600 to render immersive augmented reality or virtual reality.

Power supply 1616 may include a hard-wired power supply, a battery power supply, or a combination thereof. Power supply 1616 may provide power to computing device 1600 to enable the components of computing device 1600 to operate.

Presentation component(s) 1618 may include a display (e.g., a monitor, a touch screen, a television screen, a heads-up-display (HUD), other display types, or a combination thereof), speakers, and/or other presentation components. Presentation component(s) 1618 may receive data from other components (e.g., GPU(s) 1608, CPU(s) 1606, etc.), and output the data (e.g., as an image, video, sound, etc.).

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to code that perform particular tasks or implement particular data types. The disclosure may be practiced in a variety of system configurations, including hand-held devices, consumer electronics, general-purpose computers, more specialty computing devices, etc. The disclosure may also be practiced in distributed computing environments where tasks are performed by remote-processing devices that are linked through a communications network.

### Example

The following examples are offered to illustrate, but not to limit the claimed invention.

The example demonstrates an improved workflow of assaying 3D tumor cell samples using microfluidics-based system. The workflow may be used to obtain dynamic and static responses of cancer cells. The obtained data may be used in various implementations of training machine learning models disclosed herein, which are useful for in vitro drug discovery involving testing structurally delicate 3D cell models which can be applied to all solid tumor types.

To assay dynamic response to drug treatment effects in tumoroids, this example used a microfluidic device and flowchips, for semi-automated tumoroid assays in combination with high content imaging (FIG. 4). The System enables liquid transfers in special labware (flowchips) by applying selective pneumatic pressure. The flowchip is designed with sample wells connected to multiple adjacent wells (FIG. 4B) that can accommodate various assay reagents allowing media exchange, sample staining, wash steps, and other processing to be performed without disruption to, or loss of, the 3D sample. The bottom of the sample chamber is thin, optically clear plastic compatible with high resolution fluorescence imaging. This novel assay method using microfluidics enables automation of 3D cell-based cultures that mimic in vivo conditions, performs multi-dosing protocols and multiple media exchanges, provides gentle and convenient handling of tumoroids and organoids, and allows a wide range of assay detection modalities. This example extends use of supernatant sampling shown previously for a single end-point to a multi-point time-course of metabolite secretion that allows dynamic (temporal) profiling of tumor cell metabolism response to therapeutic compounds. This example also shows utility for immunofluorescence staining to provide biomarker characterizations.

FIG. 4 shows a Pu·MA sample processing system and flowchips used in this example. A. Flowchips in holder. The plate holder contains four flowchips and well locations conform to the SLAS/ANSI 384 well plate standard. B. Details of flowchip configuration. Each flowchip has eight test lanes with ten wells connected via microfluidics channels. The sample well has an optically clear bottom to facilitate imaging. C. Cross section of sample well showing protected sample chamber at bottom of well. The diameter of the bottom chamber is 1.2 mm with a volume of ˜1 μL. The diameter of the top of the well is 3 mm and total sample well volume is 20 μL. D. Loading flowchip plates. The Pu·MA System fits into standard size incubators. The plates are loaded using a clam-shell operation. This quickly interfaces the pneumatic manifold gasket to all 320 wells.

Data obtained from this example may be applied in breast cancer disease modeling using tumoroids formed from primary cells isolated from a patient-derived tumor, TU-BcX-4IC. This particular cell-based model was chosen in the present work as an example to show the capabilities of the assay methods described here. TU-BcX-4IC represents a rare breast cancer subtype, metaplastic breast cancer (MBC), and is classified as a triple negative breast cancer (TNBC) pathologic subtype with diverse histologic features including epithelial and mesenchymal cellular composition. This patient-derived model represents an example of a highly heterogeneous phenotype of breast cancer. TNBC tumors have an aggressive clinical presentation due to high rates of metastasis, recurrence and chemoresistance. The original patient's tumor for the TU-BcX-4IC model exhibited rapid pre-operative growth despite conventional combination therapy with adriamycin (doxorubicin), cyclophosphamide and paclitaxel. This example provides data to assess the effects of the targeted inhibitors romidepsin (primarily targets HDAC1/2) and trametinib (MEK1/2 inhibitor), as well as conventional systemic chemotherapeutics paclitaxel and cytarabine on cell viability and phenotypic change of primary derived microtissues.

### PDX Methods, Cell Lines and Tumoroid Formations

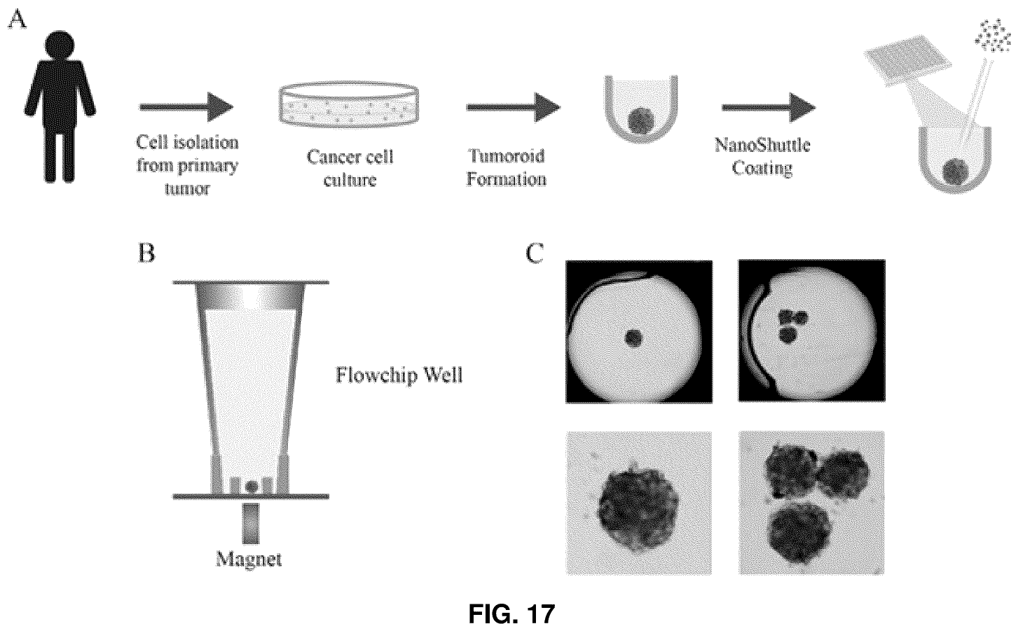

The tumor sample from TU-BcX-4IC (also hereafter denoted as ‘4IC’) implanted into SCID/Beige mice exhibited rapid tumor growth, with 14 days to reach maximal tumor volume >1000 mm3, compared to the mean length of time to maximum volume of other established TNBC PDX models. Immunohistochemistry staining of 4IC revealed both epithelial and mesenchymal histologies, and high cellularity, which was consistent throughout all serial transplants. Tumoroids were formed from cells that were isolated from the 4IC tumor and were considered primary cells (FIG. 17). Single cell suspensions containing 2000 cells per tumoroid were plated in low-attachment dishes and incubated with 5% CO2 and 37° C. conditions for 48 h until they formed tight tumoroids. 4IC cells were cultured with Advanced DMEM supplemented with glucose, NEAA, 2 mM glutamine and insulin 120 μg/L, 10% FBS (Gibco 12491-015). For metabolic assays, tumoroids were cultured with DMEM+10% dialyzed serum (2 mM glutamine, 5 mM glucose, without phenol red). Tumoroids were formed using either single well low attachment plates (ULA 384, Corning) or low attachment multicavity inserts (AggreWell 400, STEMCELL Technologies). Spheres formed by both methods exhibited high uniformity consistent with that observed with other cell models formed in 384-well low attachment plates. PDX-Os were organoids derived from patient-derived xenograft tissue generated from serial transplantation of TU-BcX-4IC intact tumor pieces in immunocompromised mice. PDX-Os were formed from cells from digested PDX tissue harvested from mice, and then 2000 cells per well were plated in a low attachment 24 well plate and incubated for 7-14 days. PDX-Os were passaged and samples were designated by passage number.

FIG. 17 shows tumoroid formation and flowchip loading. A. Cancer cells with stem-like properties are isolated from a primary tumor and cultured for 7-10 days. Cells are then loaded into low-attachment plates where they from tumoroids over a 2-5 day period. Single well or multicavity well plates can be used for this step. Optionally, tumoroids can be incubated with NanoShuttle compound to magnetize them and assist in transfer to flowchips. B. Cross section of flowchip well with tumoroid in bottom protected sample chamber. Also shown is location of magnet used to pull down and center tumoroids when using NanoShuttle. C. Transmitted light images of tumoroids loaded into flowchips. The tumoroids were formed using multicavity plates (AggreWell 400, Stemcell Technologies) and transferred with NanoShuttle which helped center them in the well.

### Microfluidic Automation

The Pu·MA System is a low to medium throughput bench-top instrument that operates inside a tissue culture incubator to perform assay protocol steps with organoids, tumoroids, and other 3D cellular structures (FIG. 4). It uses a microprocessor controlled pneumatic system to move fluid between wells via pressure differentials. A key component of the automation system is the Pu·MA System flowchip. The flowchips contain reagents and samples wells that are connected by microfluidic channels. A set of four flowchips are held in a convenient frame that conforms to the SLAS/ANSI 384 well plate standard and enables 32 samples to be tested in parallel. There is a protective chamber for micro-tissue inside the sample wells that prevents disturbance by liquid flow. Assay reagent wells are connected to the sample well via side ports allowing fluid exchange to occur without disturbing micro-tissues in the protected chamber (FIG. 4C). The flowchips are made from black cyclic olefin copolymer (COC) with a thin, optically clear COC bottom suitable for multiple assay read-outs including high resolution imaging.

Micro-tissues, media, compounds, or other reagents are pre-loaded into the flowchip by pipetting, similarly as into a regular microplate. Tumoroids were positioned into a special protected chamber at the bottom of the sample well. In some experiments, tumoroids were coated with magnetic nanoparticles (NanoShuttle, Greiner Bio One) and then positioned in the bottom of the flowchip wells using small magnets (FIG. 17B). Tumoroids were incubated with ˜0.1 μL of NanoShuttle per tumoroid for 2 h at 37° C. This helped to center the tumoroids and facilitated imaging. After sample loading, the plate was placed into the Pu·MA System located in an incubator (37° C. and 5% CO2) (FIG. 4D) and reagent exchanges were done automatically through the microfluidic channels using pre-loaded automation protocols. Multiple reagent exchanges were performed sequentially, enabling complex assay protocols to be run in an automated workflow with no manual processing steps. Typically, 8-point concentration response of two compounds were run in duplicates per plate (32 total samples). For the lactate secretion measurements, 4-point concentrations were run of three compounds in two to three replicates per plate.

### Compound Treatments

For compound screening, chemicals were prepared as 10-100 mM stock solutions in tissue culture-grade DMSO (Sigma-Aldrich, St. Louis, MO). Then compounds were diluted to appropriate concentrations in the culture media. The final concentration of DMSO in media was 0.1%. For the assays, compounds (Sigma-Aldrich) were typically tested in duplicates in a six-point dilution series. Tumoroids were transferred into flowchips and treated by performing an automated media exchange. Cells were then exposed to the various concentrations of compounds for 24 or 48 h.

### Viability and Immunofluorescence Staining

The method for imaging and high content analysis of 3D tumoroids may be used to obtain data according to various implementations. Following incubation with test compounds, tumoroids were stained for 1 h with a mixture of three dyes: 1 μM calcein AM, 3 μM of EthD-1, and 33 μM Hoechst 33342 (Thermo Fisher, Carlsbad, CA). Dyes were prepared in sterile phosphate buffered saline (PBS, Corning). Tumoroids were stained in a Pu·MA System with viability and cell surface biomarkers and imaged on ImageXpress Micro Confocal automated imaging system. Images were analyzed for percent of marker positive cells. For E-cadherin and CD44 detection tumoroids were first fixed with 4% formaldehyde solution (Sigma) for 30 min, then washed with PBS and stained with directly conjugated FITC mouse anti-E-cadherin (p/n 612130, BD Biosciences) and PE anti-human CD44 (p/n 338807, Biolegend) antibodies overnight at 1:100 dilution in the presence of 10% of Fetal Bovine Serum, then washed and imaged.

### Imaging and Analysis

Images of tumoroids were acquired using confocal automatic imaging system, ImageXpress Micro Confocal (Molecular Devices, San Jose, CA). DAPI, FITC and Texas Red filter sets were used for imaging. To image 3D organoids typically a stack of 10-15 images separated by 7-15 μm was acquired, starting at the well bottom and covering approximately the lower half of each tumoroid. Typically, a Z-stack of images covered 100-200 μm of height for each tumoroid. Image analysis was performed either in 3D using individual Z-stack images, or in 2D using the 2D Projection (maximum projection) images of confocal image stacks. Transmitted light images were used for cell culture monitoring or protocol optimization.

Images were analyzed using MetaXpress High-Content Image Acquisition and Analysis Software (Molecular Devices). Count Nuclei or and Cell Scoring application modules were used for nuclear count and live/dead assessment, respectively. A customized analysis for multiparametric measurements was done using a Custom Module Editor (CME). The custom module analysis first identified the tumoroid object using Hoechst staining. Then, individual cells were counted withing tumoroid objects by nuclear stain and viable cells were identified by presence of calcein AM or by the absence of EthD-1 signal, and dead cells were identified by presence of EthD-1 signal. In some experiments, apoptotic cells were defined using NucView 488 stain (Biotium). Measurements included counts of calcein AM positive or EthD-1 positive cells, tumoroid width, tumoroid area, volume (in 3D analysis), average intensities for calcein AM or EthD-1, counts of all nuclei, and evaluation of average nuclear size and average intensities. IC50 values were determined using 4-parameter curve fit from SoftMax® Pro 6 software (Molecular Devices) or Prism (GraphPad Software).

### Lactate Secretion Assay

The metabolic response of tumoroids to treatment was determined by measuring lactate secretion in supernatants that were collected from treated and untreated 4IC tumoroids at various timepoints of drug treatment using the Pu·MA System. Lactate data may be used to obtain temporal response profiles according to various implementations herein. Supernatant collection was done in the flowchip for each treatment condition in the following way: media with drug was transferred from an adjacent reagent well of the flowchip to the sample well with tumoroid and incubated for 3 h. After incubation, the media containing secreted lactate was transferred back to the reagent well it came from and replaced with fresh media with drugs from another well for the next 3 h treatment. The cycle repeated 5 times and resulted in collection of 5 supernatant samples. The first cycle was done with medium only (baseline secretion) followed by 4 treatment cycles for the total treatment duration of 12 h. This approach allows dynamic monitoring of lactate secretion over the course of treatment.

The collected supernatants were stored in the flowchips until the end of the Pu·MA protocol then were collected and stored at −20° C. until further processing. The supernatant samples were analyzed for lactate levels using luminescence Lactate-Glo assay (Promega). Lactate detection reagent was prepared according to the manufacturer's protocol. Supernatant samples were diluted 1:400 in PBS. 10 μL of the diluted supernatant was transferred to a solid white 384-well assay plate and 10 μL of lactate detection reagent was added to each well. Plates were incubated for 60 min at room temperature. Luminescence was measured using GloMax plate reader (Promega). Each sample was measured in duplicate. Good luminescence signal levels and signal-to-noise ratios were achieved for this assay from single tumoroid samples.

### Luminescence Viability Assays

Two luminescence-based viability assays were used in conjunction with the metabolite assays: RealTime-Glo MT [[18]] (RT-Glo, Promega) and CellTiter-Glo 3D (CTG, Promega). All incubations and luminescence measurements for these assays were done within the flowchips. RT-Glo assay was performed prior to the treatment initiation to assess tumoroid size and viability. RT-Glo reagent was prepared in culture media according to the manufacturer's protocol. 20 μL of the prepared RT-Glo solution was added directly to the sample well in the flowchips containing the tumoroid. After that flowchips were incubated for 2 h at 37° C. Luminescence signal was measured in a plate reader (GloMax, Promega). CTG 3D assay was done at the end of the incubation of tumoroids with compounds. Flowchips and CTG 3D reagent were equilibrated to room temperature. After that 10 μL of media was removed from the sample well with tumoroid and replaced with 10 μL of the CTG 3D assay reagent. The flowchip plate was incubated for 40 min at room temperature and then luminescence signal was measured in a plate reader.

Data generated by various assays above may be used to generate static and dynamic response data and TU-BcX-4IC tumor outcome data. They may also be applied to generate data for other types of diseases. The generated data may be used to train and apply machine learning models to discover therapeutics for treating diseases according to some implementations disclosed herein.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

1. A method of treating a plurality of cell samples of a model of a disease, comprising:
loading each cell sample of the plurality of cell samples into a first microfluidic well of a microfluidic flowchip, wherein the microfluidic flowchip comprises one or more networks of microfluidic wells connected by microfluidic channels, and wherein the microfluidic flowchip is controlled by one or more processors configured to automate fluid flow in the microfluidic flowchip;
exposing each cell sample of the plurality of cell samples to a therapeutic of a plurality of therapeutics, wherein each therapeutic is selected from a group consisting of one or more compounds, one or more cells, one or more physical conditions, or any combinations thereof, and wherein the plurality of therapeutics are different in at least one aspect;
repeatedly measuring over a period of time at least one dynamic response of each cell sample after the cell sample is exposed to the therapeutic, thereby producing at least one temporal response profile for each cell sample comprising a plurality of measurements of the dynamic response obtained at a plurality of points in the period of time;
assaying one or more outcome phenotypes of each cell sample after the cell sample is exposed to the therapeutic; and
training at least one machine learning model using training data representing: (a) one or more differences among the plurality of therapeutics, (b) the at least one temporal response profile for each cell sample, and (c) the one or more outcome phenotypes of each cell sample, wherein the at least one machine learning model receives as input one or more variables representing the one or more differences and the at least one temporal response profile and provides as output one or more variables representing the one or more outcome phenotypes.

2. The method of claim 1, wherein the at least one machine learning model comprises a first machine learning model that receives as input one or more variables representing the one or more differences and provides as output at least one variable representing the at least one temporal response profile, and a second machine learning model that receives as input the variable representing the temporal response profile and provides as output the one or more variables representing the one or more outcome phenotypes.

3. The method of claim 1, wherein the at least one machine learning model comprises a machine learning model that receives as input one or more variables representing the one or more differences and the at least one temporal response profile and provides as output one or more variables representing the one or more outcome phenotypes.

4. The method of claim 1, wherein a first machine learning model of the at least one machine learning model generates one or more intermediate variables as output, and a second machine learning model of the at least one machine learning model receives as input the one or more intermediate variables and provides as output the one or more variables representing the one or more outcome phenotypes.

5. The method of claim 4, wherein each of the one or more intermediate variables is selected from a group consisting of: a variable representing the at least one temporal response profile, a T-cell Functional Response Score (TFRS), a tumor response score, a cell type specific response score, a therapeutic treatment response score, a therapeutic sensitivity score, a therapeutic resistance score, a latent variable, a first derivative of the temporal response profile, a second derivative of the temporal response profile, an IC50 value, an EC50 value, a transition state expression transient, and any combinations thereof.

6. (canceled)

7. The method of claim 1, wherein the at least one machine learning model comprises one or more models selected from a group consisting of: a neural network, a convolutional neural network (CNN), an autoencoder, a variational autoencoder (VAE), a regression model, a linear model, a non-linear model, a support vector machine, a decision tree model, a random forest model, an ensemble model, a Bayesian model, a naïve Bayes model, a k-means model, a k-nearest neighbors model, a principal component analysis, a Markov model, and any combinations thereof.

8. The method of claim 1, further comprising: providing values of the one or more variables representing the one or more differences for one or more new therapeutics to the at least one trained machine learning model to predict the one or more outcome phenotypes for the one or more new therapeutics, wherein the one or more new therapeutics are different from the plurality of therapeutics.

9. The method of claim 8, further comprising: exposing a cell sample of the model of the disease to a new therapeutic predicted to result in values of the one or more outcome phenotypes meeting one or more criteria.

10. (canceled)

11. The method of claim 1, wherein the at least one machine learning model receives as input one or more static response variables each representing a static response of the cell sample measured at one point of time after the cell sample is exposed to the therapeutic.

12. The method of claim 1, wherein the at least one machine learning model receives as input one or more pre-treatment variables each representing a pre-treatment phenotype of the cell sample measured before the cell sample is exposed to the therapeutic.

13. (canceled)

14. The method of claim 1, wherein each aspect of the at least one aspect is selected from a group consisting of: an identity of a therapeutic, a presence of a therapeutic, a structural component of a therapeutic, a functional component of a therapeutic, a dosage of a therapeutic, a concentration of a therapeutic, a time when a therapeutic is applied, and any combinations thereof.

15. The method of claim 1, wherein each cell sample comprises a three-dimensional cell sample comprising one or more cells, a tumoroid, an organoid, a spheroid, a multicellular spheroid, an ellipsoid, or a three-dimensional sample comprising different cell types.

16. (canceled)

17. (canceled)

18. (canceled)

19. The method of claim 1, wherein the at least one dynamic response comprises a change in an item selected from a group consisting of: cellular function, biomarker expression, cellular secretion, cellular structure, protein expression, protein cellular localization, protein-protein interaction, cell-cell interaction, cell-extracellular matrix interaction, cell signaling, cell death process, cell viability, and any combinations thereof.

20. The method of claim 1, wherein the at least one dynamic response comprises a change in an item selected from a group consisting of: cytokines, chemokines, growth factors, and any combinations thereof.

21. (canceled)

22. The method of claim 1, wherein the at least one dynamic response comprises a metabolic response.

23. (canceled)

24. The method of claim 1, wherein the at least one dynamic response comprises an immune response.

25. (canceled)

26. The method of claim 1, wherein the at least one dynamic response comprises a cancer resistance response.

27. (canceled)

28. The method of claim 1, wherein the at least one dynamic response comprises a inflammation response.

29. The method of claim 1, wherein the one or more outcome phenotypes is selected from a group consisting of: number of cells, number of live cells, number of dead cells, cell proliferative index, apoptosis, integrity of cells, shape of cells, size of cells, size of sample, cell-cell distance, distance between cell types, shape, size, area, volume, perimeter, roundness/circularity of a three-dimensional sample, and any combinations thereof.

30. The method of claim 1, wherein loading each cell sample comprises: coating the cell sample with magnetic nanoparticles and immobilizing the cell sample in the first microfluidic well using a magnet.

31. The method of claim 1, exposing each cell sample of the plurality of cell samples to the therapeutic of a plurality of therapeutics comprises: loading each therapeutic into each of second microfluidic wells and transferring each therapeutic from each second microfluidic well to each first microfluidic well.

32. The method of claim 1, wherein the at least one dynamic response is measured in situ in the first microfluidic well.

33. The method of claim 1, wherein repeatedly measuring the at least one dynamic response comprises transferring a supernatant from the first microfluidic well to a third microfluidic well and assaying the supernatant in the third microfluidic well.

34. The method of claim 1, the period of time comprises a period of at least 5, 10, 15, 20, 25, 30, 35, 40, 45, 50, 55 minutes, 1, 2, 3 4, 5, 6, 7, 8, 9, 12, 15, 18, 21 or 24 hours, or 1, 2, 3, 4, 5, 6, or 7 days, or 1, 2, 3, or 4 weeks and the plurality of points in the period of time comprises at least 4, 10, 20, 30, 40, 50, 60, 70, 80, 90, 100, 200, 500, 1000 points in the period of time.

35. (canceled)

36. The method of claim 1, wherein to train the at least one machine learning model comprises:
training a first forward machine learning model that receives as input the one or more variables corresponding to the one or more differences among the plurality of therapeutics and generates first model output data corresponding to the at least one temporal response profile; and
training a second forward machine learning model that receives as input the training data corresponding to the at least one temporal response profile and generates second model output data corresponding to the one more outcomes.

37. The method of claim 36, wherein the first forward machine learning model or the second forward machine learning model is selected from a group consisting of: a neural network, a convolutional neural network (CNN), an autoencoder, a variational autoencoder (VAE), a regression model, and any combinations thereof.

38. (canceled)

39. (canceled)

40. The method of claim 36, wherein the first or second forward machine learning model receives as further input one or more static response variables each corresponding to a static response of a cell sample measured at one point of time after the cell sample is exposed to the therapeutic.

41. The method of claim 36, wherein the first or second forward machine learning model receives as further input one or more pre-therapeutic variables each corresponding to a measurement of a cell sample taken before the cell sample is exposed to the therapeutic.

42. (canceled)

43. The method of claim 36, wherein:
the second forward machine learning model comprises a variational autoencoder (VAE) and at least one of a regression model or a CNN;
the VAE takes the at least one temporal response profile as input and generates latent variables in a latent space; and
the regression model or CNN takes the latent variables as input and provides the one or more outcome phenotypes as output.

44. (canceled)

45. The method of claim 36, wherein the first forward machine learning model provides as further output one or more variables selected from a group consisting of: a T-cell Functional Response Score (TFRS), a tumor response score, a cell type specific response score, a therapeutic treatment response score, a therapeutic sensitivity score, a therapeutic resistance score, a latent variable, a first derivative of the temporal response profile, a second derivative of the temporal response profile, an IC50 value, an EC50 value, and a transition state expression transient.

46. The method of claim 45, wherein the second forward machine learning model receives as further input one or more variables selected from the group consisting of: a T-cell Functional Response Score (TFRS), a tumor response score, a cell type specific response score, a therapeutic treatment response score, a therapeutic sensitivity score, a therapeutic resistance score, a latent variable, a first derivative of the temporal response profile, a second derivative of the temporal response profile, an IC50 value, an EC50 value, and a transition state expression transient.

47. The method of claim 36, wherein the one or more processors is further configured to:
train a first backward machine learning model that receives as input data corresponding to the at least one temporal response profile and generates third model output data corresponding to the one or more differences among the plurality of therapeutics; and
train a second backward machine learning model that receives as input data representing the one more outcome phenotypes and generates fourth model output data corresponding to the at least one temporal response profile.

48. The method of claim 47, further comprising:
receiving test data representing a desired outcome phenotype;
generating, using the test data representing the desired outcome phenotype and the trained second backward machine learning model, at least one desired temporal response profile; and
predicting, using the at least one desired temporal response profile and the trained first backward machine learning model, a new therapeutic or a plurality of new therapeutics corresponding to the at least one desired temporal profile and the desired outcome.

49. (canceled)

50. The method of claim 47,
wherein:
the second forward machine learning model comprises a variational autoencoder (VAE) and at least one of a CNN or a regression model;
the VAE takes the at least one temporal response profile as input and generates latent variables in a latent space; and
the CNN or regression model takes the latent variables as input and provides the one or more outcome phenotypes as output;
further comprising:
identifying a set of desired latent variables that the CNN or regression model takes as input while producing one or more desired outcome phenotypes as output;
generating at least one desired temporal response profile using the one or more desired outcome phenotypes and the VAE; and
generating output data corresponding to a desired new therapeutic using the at least one desired temporal response profile and the trained first backward machine learning model.

51. (canceled)

52. The method of claim 47, wherein the first backward machine learning model or the second backward machine learning model is selected from a group consisting of: a neural network, a convolutional neural network (CNN), an autoencoder, a variational autoencoder (VAE), a regression model, a conditional generative adversarial network (cGAN), and any combinations thereof.

53. (canceled)

54. (canceled)

55. The method of claim 47, wherein the first backward machine learning model receives as further input or generates as further output one or more static response variables each corresponding to a static response of a cell sample measured at one point of time after the cell sample is exposed to the therapeutic.

56. (canceled)

57. The method of claim 47, wherein the first backward machine learning model receives as further input or generates as further output one or more pre-therapeutic variables each corresponding to a measurement of a cell sample taken before the cell sample is exposed to the therapeutic.

58. (canceled)

59. (canceled)

60. (canceled)

61. The method of claim 36, further comprising:
receiving test data representing at least one new therapeutic, the at least one new therapeutic being different from the plurality of therapeutics;
generating, using the test data representing the at least one new therapeutic and the trained first forward machine learning model, at least one temporal response profile for each new therapeutic; and
predicting, using the at least one temporal response profile for each new therapeutic and the trained second forward machine learning model, the one more outcome phenotypes of a cell sample after the cell sample is exposed to each new therapeutic.

62. (canceled)

63. A system for training a machine learning model comprising one or more processors and system memory, the one or more processors being configured to:
receive training data representing: (a) one or more differences among a plurality of therapeutics or stimuli, (b) at least one temporal response profile for each cell sample of a plurality of cell samples of a model of a disease, the at least one temporal response profile is obtained after the cell sample is exposed to a therapeutic of the plurality of therapeutics or stimuli, and (c) one or more phenotypes of each cell sample of the plurality of cell samples after the cell sample is exposed to the therapeutic or stimuli; and
train at least one machine learning model using the training data, wherein the at least one machine learning model receives as input one or more variables representing the one or more differences and the at least one temporal response profile and provides as output one or more variables representing the one or more phenotypes.

64. (canceled)

65. A non-transitory machine readable medium having stored thereon program code that, when executed by one or more processors of a computer system, causes the computer system to train a machine learning model, said program code comprising code for:
receiving training data representing: (a) one or more differences among a plurality of therapeutics or stimuli, (b) at least one temporal response profile for each cell sample of a plurality of cell samples of a model of a disease, the at least one temporal response profile is obtained after the cell sample is exposed to a therapeutic of the plurality of therapeutics or stimuli, and (c) one or more phenotypes of each cell sample of the plurality of cell samples after the cell sample is exposed to the therapeutic or stimuli; and
training at least one machine learning model using the training data, wherein the at least one machine learning model receives as input one or more variables representing the one or more differences and the at least one temporal response profile and provides as output one or more variables representing the one or more phenotypes.

66.-102. (canceled)
